(19) United States
(12) Patent Application Publication
(10) Pub. No.: US 2024/0009469 A1
(43) Pub. Date: Jan. 11, 2024

(54) SYSTEM FOR DETERMINING AN ESTIMATE OF BATTERY CAPACITY FOR AN IMPLANTABLE DEVICE

(71) Applicant: Axonics, Inc., Irvine, CA (US)

(72) Inventors: Prabodh MATHUR, Laguna Niguel, CA (US); Charles Borlase, Lake Forest, CA (US); Pete Peterson, Irvine, CA (US)

(73) Assignee: Axonics, Inc., Irvine, CA (US)

(21) Appl. No.: 18/219,613

(22) Filed: Jul. 7, 2023

Related U.S. Application Data
(60) Provisional application No. 63/359,463, filed on Jul. 8, 2022.

(51) Int. Cl. A61N 1/378; A61N 1/372; A61N 1/02

(52) U.S. Cl. CPC A61N 1/378; A61N 1/37235; A61N 1/025

(57) ABSTRACT

A system for determining an estimate of a battery capacity for the battery of an implantable device that is used over a period of time. The system includes an external device having a processor that is configured to communicate with the implantable device. During the initial phase, the processor is configured to determine the estimate of the battery capacity using battery energy consumption data and a battery capacity at the beginning of use. During the latter phase, the processor is configured to determine the estimate of the battery capacity based on the battery voltage. The system may include an intermediate stage and, during the intermediate phase, the processor is configured to determine the estimate of the battery capacity based on the battery energy consumption data and the battery voltage.

### CROSS-REFERENCE TO RELATED APPLICATION

This application claims priority to and the benefit of U.S. Provisional Patent Application No. 63/359,463 filed Jul. 8, 2022, which is incorporated by reference herein in its entirety.

### GENERAL DESCRIPTION

This disclosure relates generally to implantable medical devices such as neurostimulation treatment systems and, in particular, to methods and systems for determining an estimate of the remaining capacity of a battery for implanted medical devices. Remaining capacity is the amount of energy in the battery that is available for use at a given point in time.

Treatments with implantable medical devices such as neurostimulation systems have become increasingly common in recent years. For example, stimulation systems often make use of an array of electrodes to treat one or more target nerve structures. The electrodes are often mounted together on a multi-electrode lead, and the lead implanted in tissue of the patient at a location that is intended to result in electrical coupling of the electrode to the target nerve structure, typically with at least a portion of the coupling being provided via intermediate tissues. Other approaches may also be employed, for example, with one or more electrodes attached to the skin overlying the target nerve structures, implanted in cuffs around a target nerve, or the like. Regardless, the physician will typically seek to establish an appropriate treatment protocol by varying the electrical stimulation that is applied to the electrodes.

The nerve tissue structures of different patients may be quite different. The electrical properties of the tissue structures surrounding a target nerve structure may also be quite different among different patients, and the neural response to stimulation may be markedly dissimilar, with an electrical stimulation pulse pattern, pulse width, frequency, and/or amplitude that is effective to affect a body function of one patient and potentially imposing significant discomfort or pain, or having limited effect, on another patient. Even in patients where implantation of a neurostimulation system provides effective treatment, frequent adjustments and changes to the stimulation protocol are often required before a suitable treatment program may be determined, often involving repeated office visits and significant discomfort for the patient before efficacy is achieved. Such implanted neurostimulation devices often include a battery that meets the power demands of the device in performing stimulation as well as control and telemetry functions.

Accordingly, the lifetime and battery life of such devices is limited and can vary considerably depending on usage. Previously, implanted systems with non-rechargeable batteries were generally replaced every five to seven years. More recently, implanted systems utilize batteries that require replacement every 10 years or more. Since replacement requires additional surgery, patient discomfort, and significant costs to healthcare systems, it is important for both patient and clinician to accurately estimate the battery capacity periodically over the lifetime of use of the battery. Currently, there is often uncertainty as to the remaining life of the battery, particular since parameters of the battery may remain generally consistent for much of the life of the battery and degrade rapidly toward end of life. Current systems and methods of determining battery capacity often do not provide consistent and accurate assessments of remaining battery life. Therefore, it is desirable to provide more accurate and reliable predictions and/or estimates of the remaining capacity of the batter contained in implantable devices, particularly for neurostimulation systems, which can considerably improve patient's comfort and resolve uncertainties in planning and timing of device replacement.

### SUMMARY

Aspects of the present disclosure relate to a method, a system, and an apparatus for determining a remaining battery capacity of a battery of an implantable device such as an implantable pulse generator (IPG). Typically, the battery is a non-rechargeable primary cell battery, for example, a lithium manganese dioxide (Li—MnO2).

One aspect of the present disclosure relates to methods of determining an estimated remaining battery capacity of a battery in an implantable device over a useful life of the device. Such methods can include: establishing communication between the implantable device and an external device (e.g., a clinician programmer, a patient remote, or other devices), and receiving information from the implantable device including battery energy consumption data (e.g., data set of energy consumed by specific loads on the battery or cumulative value of energy consumed by loads on the battery) and a voltage of the battery. Based on the received information, the estimated remaining battery capacity is determined. In some embodiments, a method is used that can employ one or more technique or equations to determine the estimated remaining battery capacity. In some embodiments, the technique or equations depend on a phase of the time period (e.g., initial phase, intermediate, tertiary phase, latter phase) of the useful life of the battery.

In some embodiments, during an initial phase of use, the estimated remaining battery capacity is determined from data related to cumulative battery energy consumption relative to a total capacity of the battery at full charge. During an intermediate phase of use, the estimated remaining battery capacity is based on a combination of the battery energy consumption data and the battery voltage at the time that the estimated battery capacity is determined. During a tertiary phase of use, the determination of the estimated remaining battery capacity is based on the voltage. In some embodiments, the methods include: estimating remaining battery capacity from cumulative battery energy consumption during an initial phase and estimating battery capacity based on voltage during a latter phase. In some embodiments, the latter phase is directly after the initial phase, while in other embodiments, the latter phase is a tertiary or final phase.

In some embodiments, during the intermediate phase, a first estimate result based on battery energy consumption data and a second estimate result based on voltage are combined linearly. In some embodiments, the intermediate phase is when the voltage is within a voltage range between an upper battery voltage threshold (e.g., 95-99% of nominal open circuit voltage) and a lower battery voltage threshold (e.g., 85-95% of nominal voltage). The nominal voltage may be provided by the manufacturer of the battery (e.g., 3.1V for the Litronik Li S battery described below) and generally corresponds to the open circuit voltage of the new and unused battery. In some embodiments, the battery energy consumption data set and the voltage may be combined linearly such that first estimate result battery based on energy consumption data is fully weighted upon commencement of the intermediate phase and the second estimate result based on voltage is fully weighted at completion of the intermediate phase.

In some embodiments, the tertiary phase occurs when the voltage is below the lower voltage threshold (e.g., between 85-95% of nominal voltage). In some embodiments, the estimate during the tertiary phase is determined from the voltage based on a polynomial equation derived from characterization data of the battery discharge.

In some embodiments, the initial phase may include a first subphase and a second subphase. During the first subphase, the estimated remaining battery capacity is determined from battery consumption data when greater than an upper capacity threshold (e.g., between 70%-80% capacity, about 75%). During the second subphase, when the estimated capacity is below the upper capacity threshold the estimate is determined from battery consumption and floored (i.e., has a minimum value) at a lower capacity threshold (e.g., between than 40%-50% capacity, about 46%). Also, during the second subphase, the voltage may be greater than the upper battery voltage threshold. During the initial phase, the remaining battery capacity may be determined as a function of the battery consumption data set (e.g., a dead reckoning technique).

Another aspect pertains to an external device (e.g., a clinician programmer, a patient remote, or other devices) configured to determine a remaining battery capacity according to the method above. In some embodiments, the external device is communicably coupleable with the implantable device. The external device includes a graphical user interface configured to facilitate programming and monitoring of the implantable device, and one or more processors operably coupled with a memory having recorded thereon executable instructions to perform the methods described herein. For example, the instructions may be configured for establishing communication between the implantable device and the programmer, and receiving information from the implantable device including battery energy consumption data and a voltage of a battery of the implantable device, and determining, based on the received information, an estimated remaining battery capacity. During an initial phase of use, the estimated remaining battery capacity is determined from a cumulative battery energy consumption relative a total capacity at full charge. During an intermediate phase of use, the estimated remaining battery capacity is determined based on a combination of the battery energy consumption data and the voltage. During a tertiary phase of use, the estimated remaining battery capacity is determined based on the voltage. In some embodiments, the external device is configured to: estimate remaining battery capacity from cumulative battery energy consumption during an initial phase and estimate battery capacity based on voltage during a latter phase. In some embodiments, the latter phase is directly after the initial phase, while in other embodiments, the latter phase is a tertiary or final phase.

### BRIEF DESCRIPTION OF THE DRAWINGS

FIG. 1 schematically illustrates an exemplary nerve stimulation system, which includes a clinician programmer and a patient remote used in positioning and/or programming of both a trial neurostimulation system and a permanently implanted neurostimulation system.

FIGS. 2A-2C show diagrams of the nerve structures along the spine, the lower back and sacrum region.

FIG. 3 shows an example of a fully implanted neurostimulation system.

FIG. 4 shows an example of a neurostimulation system having an implantable stimulation lead, an implantable pulse generator, and an external charging device.

FIGS. 5A and 5B show detail views of an implantable pulse generator (IPG) and associated components for use in a neurostimulation system.

FIG. 6 shows a schematic illustration of one embodiment of the architecture of an IPG.

FIG. 7 shows an example battery discharge curve plotting battery voltage as a function of remaining battery capacity including differing phases utilized in determining remaining battery capacity in accordance with some embodiments.

FIG. 8 shows an example of the battery discharge curve with the axes reverse to show remaining capacity (as a percentage of full capacity) as a function of voltage and further shows differing phases and sub-phases utilized in determining remaining battery capacity in accordance with some embodiments.

FIGS. 9 and 10 show example flow charts of methods for estimating a remaining battery capacity of an implantable device in accordance with some embodiments.

### DETAILED DESCRIPTION

The present application relates to neurostimulation treatment systems and associated implantable devices and programmer devices, in particular methods and systems for determining a remaining battery capacity estimate of a battery for an implantable device. In the example embodiment described herein, the system is a sacral nerve stimulation treatment system configured to treat overactive bladder (“OAB”) and relieve symptoms of bladder related dysfunction. However, it is appreciated that the devices, systems and methods disclosed herein may also be utilized for a variety of neuromodulation uses, such as fecal dysfunction, and the treatment of pain or other indications, such as movement or affective disorders, or for any implantable medical device that is powered by a battery.

Neurostimulation (or neuromodulation as may be used interchangeably herein) treatment systems, such as any of those described herein, may be used to treat a variety of ailments and associated symptoms, such as acute pain disorders, movement disorders, affective disorders, as well as bladder related dysfunctions. Examples of pain disorders that may be treated by neurostimulation include failed back surgery syndrome, reflex sympathetic dystrophy or complex regional pain syndrome, causalgia, arachnoiditis, and peripheral neuropathy.

Movement disorders include muscle paralysis, tremor, dystonia and Parkinson's disease. Affective disorders include depression, obsessive-compulsive disorder, cluster headache, Tourette's syndrome and certain types of chronic pain. Bladder-related dysfunctions include but are not limited to OAB, urge incontinence, urgency-frequency, and urinary retention. OAB is one of the most common urinary dysfunctions and is characterized by the presence of bothersome urinary symptoms, including urgency, frequency, nocturia and urge incontinence, and can include any of these symptoms alone or in combination.

Neurostimulation methods include sacral neuromodulation (SNM). SNM is an established therapy that provides a safe, effective, reversible, and long-lasting treatment option for the management of OAB. SNM therapy involves the use of mild electrical pulses to stimulate the sacral nerves located in the lower back. Electrodes are placed next to a sacral nerve, usually at the S3 level, by inserting a lead into the corresponding foramen of the sacrum. The lead is inserted subcutaneously and is subsequently attached to an implantable pulse generator (IPG), also referred to herein as an implantable neurostimulator or a neurostimulator.

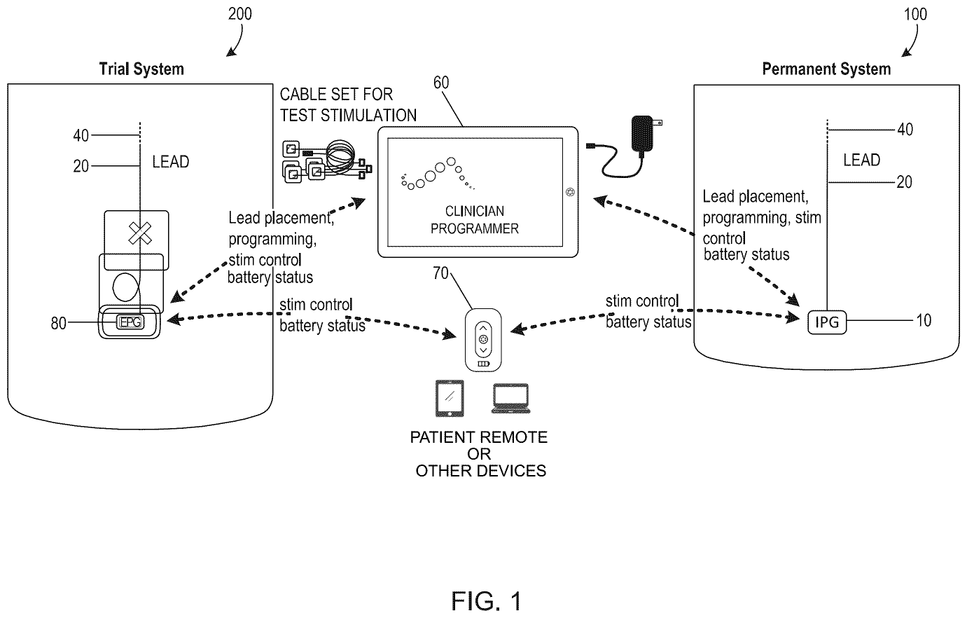

FIG. 1 schematically illustrates an exemplary neurostimulation system, which includes both a trial neurostimulation system 200 and a permanently implanted neurostimulation system 100. An external pulse generator (EPG) 80 and an implantable pulse generator (IPG) 10 are each compatible with and wirelessly communicate with a clinician programmer 60 and a patient remote 70, which are used in positioning and/or programming the trial neurostimulation system 200 and/or permanently implanted system 100 after a successful trial. The clinician programmer can include specialized software, specialized hardware, or both, to aid in lead placement, programming, re-programming, stimulation control, and/or parameter setting, as well determining remaining battery capacity estimate. In addition, the patient remote 70 can be used with one or both of the IPG and the EPG to provide at least some control over stimulation (e.g., initiating a pre-set program, increasing or decreasing stimulation), and/or to monitor battery status.

In one aspect, the clinician programmer 60 is used by a physician to adjust the settings of the EPG and/or IPG while the lead is implanted within the patient. The clinician programmer may be a tablet computer used by the clinician to program the IPG, or to control the EPG during the trial period. The patient remote 70 can allow the patient to turn the stimulation on or off, or to vary stimulation from the IPG while implanted, or from the EPG during the trial phase.

In another aspect, the clinician programmer 60 has a control unit which can include a microprocessor and specialized computer-code instructions for implementing methods and systems for use by a physician in deploying the treatment system, setting up treatment parameters, as well as periodic assessments of the IPG, including the remaining battery capacity estimates described herein. The clinician programmer generally includes a user interface which may be a graphical user interface.

The electrical pulses generated by the EPG and IPG are delivered to one or more targeted nerves through one or more electrodes at or near a distal end of each of one or more leads. The leads may vary in size, shape, and materials and may be tailored for a specific treatment application. For an SNM system, the lead is of a suitable size and length to extend from the IPG and through one of the foramen of the sacrum to a targeted sacral nerve. However, the leads and/or the stimulation programs may vary according to the nerves being targeted.

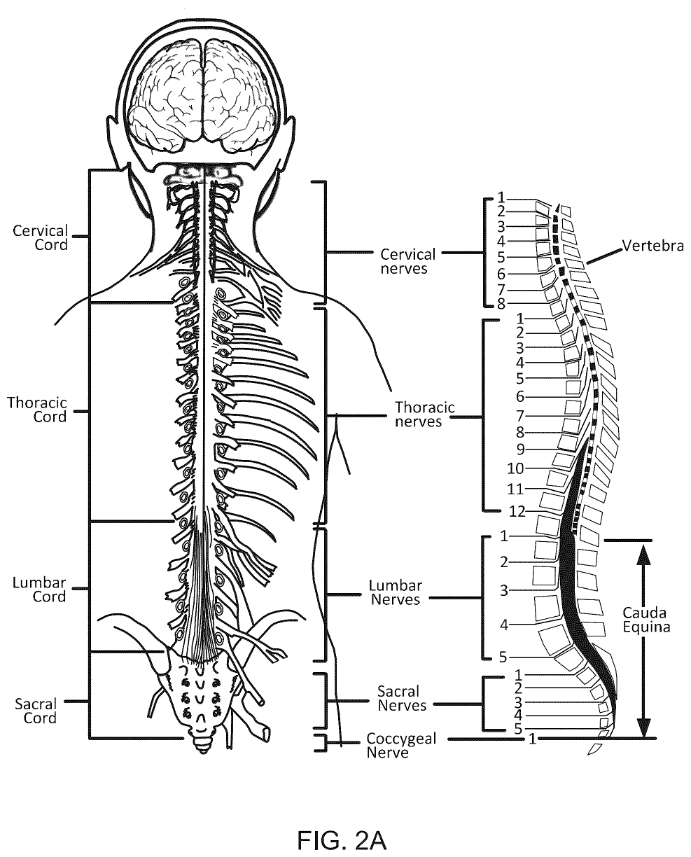

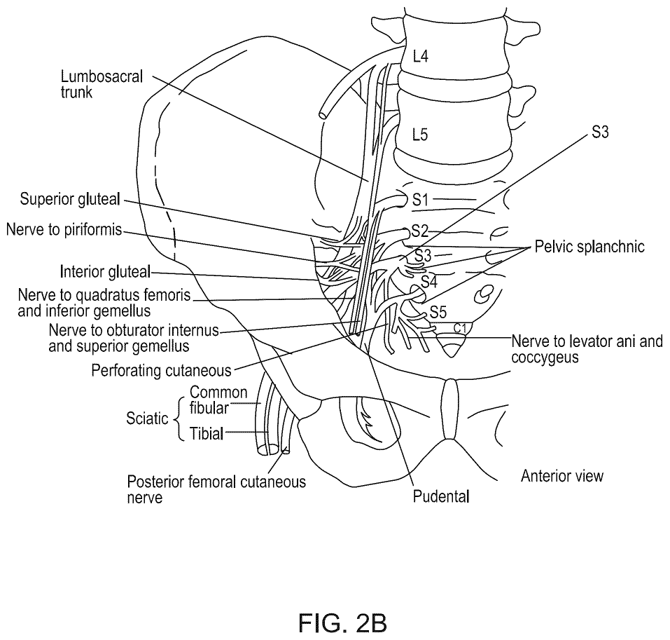

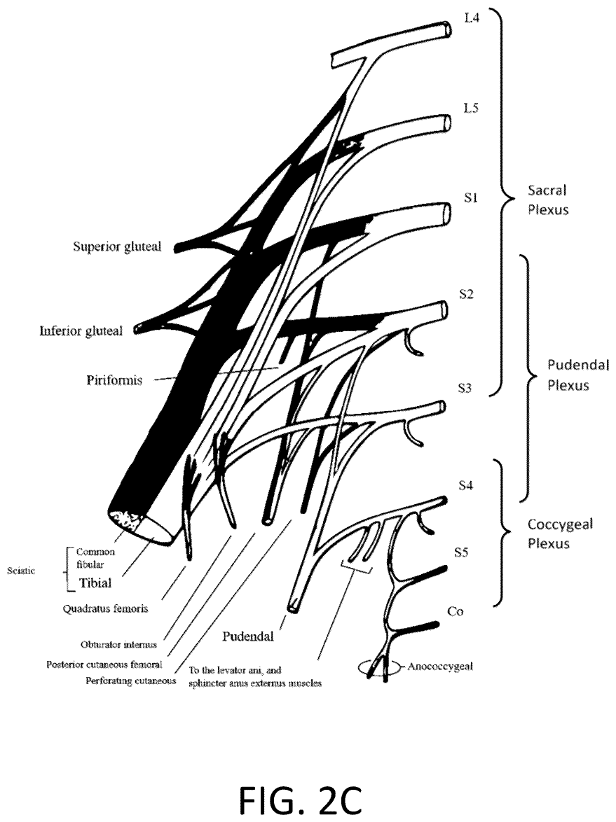

FIGS. 2A-2C show diagrams of various nerve structures of a patient, which may be targeted in neurostimulation treatments. FIG. 2A shows the different sections of the spinal cord and the corresponding nerves within each section. The spinal cord is a long, thin bundle of nerves and support cells that extend from the brainstem along the cervical cord, through the thoracic cord and to the space between the first and second lumbar vertebra in the lumbar cord. Upon exiting the spinal cord, the nerve fibers split into multiple branches that innervate various muscles and organs transmitting impulses of sensation and control between the brain and the organs and muscles. Since certain nerves may include branches that innervate certain organs and branches that innervate certain muscles, stimulation of the nerve at or near the nerve root near the spinal cord can stimulate the nerve branch that innervates the targeted organ or muscle.

FIG. 2B shows the nerves associated with the lower back section, in the lower lumbar cord region where the nerve bundles exit the spinal cord and travel through the sacral foramina of the sacrum. In some embodiments, the lead is advanced through the foramen until the electrodes are positioned at the anterior sacral nerve root, while the anchoring portion of the lead are generally disposed dorsal of the sacral foramen through which the lead passes to anchor the lead. FIG. 2C shows detail views of the nerves of the lumbosacral trunk and the sacral plexus, in particular, the S1-S5 nerves of the lower sacrum. The S3 sacral nerve is of particular interest for treatment of bladder related dysfunction, and in particular OAB.

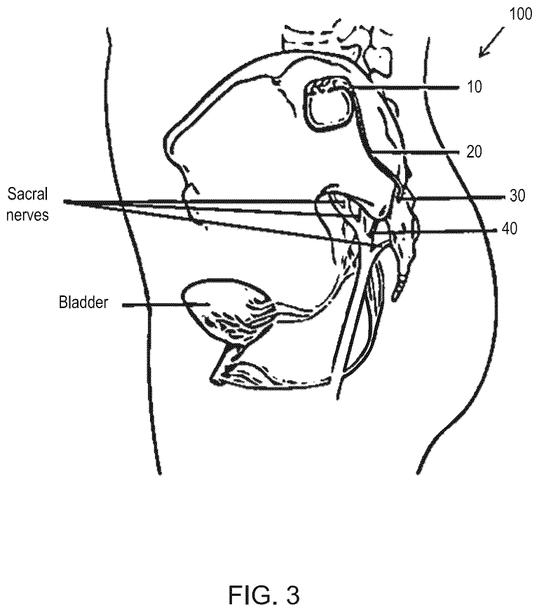

FIG. 3 schematically illustrates an example of a fully implanted neurostimulation system 100 adapted for sacral nerve stimulation. Neurostimulation system 100 includes an IPG implanted in a lower back region and connected to a lead 20 extending through the S3 foramen for stimulation of the S3 sacral nerve. The lead is anchored by a tined anchor portion 30 that maintains a position of a set of neurostimulation electrodes 40 along the targeted nerve, which in this example, is the anterior sacral nerve root S3 which enervates the bladder so as to provide therapy for various bladder-related dysfunctions. While this embodiment is adapted for sacral nerve stimulation, it is appreciated that similar systems may be used to either stimulate a target peripheral nerve or the posterior epidural space of the spine for the treatment of other indications.

In the embodiment depicted in FIG. 3, the implantable neurostimulation system 100 includes a controller in the IPG having one or more pulse programs, plans, or patterns that may be pre-programmed or created as discussed above. In some embodiments, these same properties associated with the IPG may be used in an EPG of a partly implanted trial system used before implantation of the permanent neurostimulation system 100.

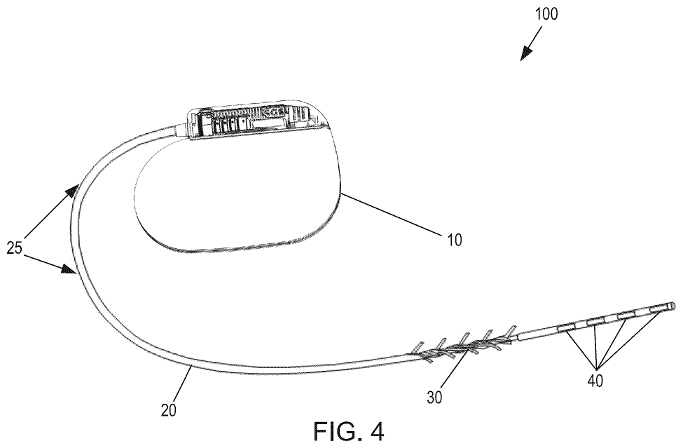

FIG. 4 illustrates an example neurostimulation system 100 that is fully implantable and adapted for sacral nerve stimulation treatment. The implantable system 100 includes an IPG 10 that is coupled to a neurostimulation lead 20 that includes a group of neurostimulation electrodes at a distal end of the lead. The lead includes a lead anchor portion 30 with a series of tines extending radially outward to anchor the lead and maintain a position of the lead 20 after implantation. The lead 20 may further include one or more radiopaque markers 25 to assist in locating and positioning the lead using visualization techniques such as fluoroscopy. In some embodiments, the IPG provides monopolar or bipolar electrical pulses that are delivered to the targeted nerves through one or more electrodes, typically four electrodes. In sacral nerve stimulation, the lead is typically implanted through the S3 foramen as described herein.

The system may further include a patient remote 70 and clinician programmer 60, each configured to wirelessly communicate with the implanted IPG during long-term therapy, or with the EPG during a trial. The clinician programmer 60 may be a tablet computer used by the clinician to program the IPG and the EPG. The patient remote may be a battery-operated, portable device that utilizes radio-frequency (RF) signals to communicate with the EPG and IPG and allows the patient to adjust the stimulation levels, check the status of the IPG battery level, and/or to turn the stimulation on or off. The IPG may utilized RF polling at differing scan rates in order to facilitate communication sessions with the clinician programmer and patient remote.

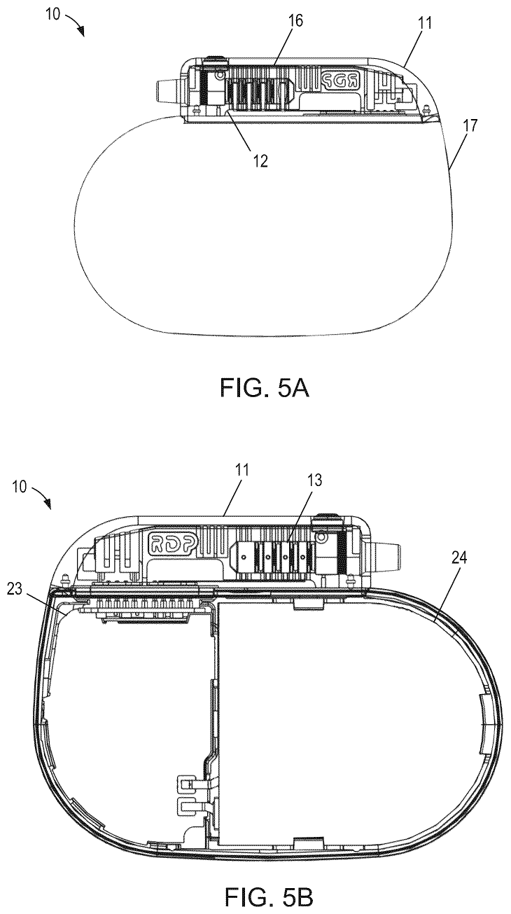

FIGS. 5A and 5B show detail views of the IPG 10 and its internal components. In some embodiments, the pulse generator may generate one or more non-ablative electrical pulses that are delivered to a nerve to produce a desired effect, for example to control pain or inhibit, prevent, or disrupt neural activity for the treatment of OAB or bladder related dysfunction. In some applications, the pulses having a pulse amplitude in a range between 0 mA to 1,000 mA, 0 mA to 100 mA, 0 mA to 50 mA, 0 mA to 25 mA, and/or any other or intermediate range of amplitudes may be used. The pulse generator may include a controller (e.g. processor) and/or memory adapted to provide instructions to and receive information from the other components of the implantable neurostimulation system. The processor may include a microprocessor, such as a commercially available microprocessor from Intel® or Advanced Micro Devices, Inc.®, or the like.

One or more properties of the electrical pulses may be controlled by a processor or controller of the IPG or EPG. These properties may include, for example, the frequency, strength, pattern, duration, or other aspects of the timing and magnitude of the electrical pulses. In addition, the controller may vary the voltage and current used to generate the pulse. The controller may establish a repeatable pattern or program of pulses applied to the electrodes. For example, the system may provide for the selection of a predetermined electrical pulse program, plan, or pattern from one or more available options. In one aspect, the IPG 10 includes a controller, also referred to herein as a processor or microprocessor, having one or more pulse programs, plans, or patterns that may be created and/or pre-programmed. In some embodiments, the IPG may be programmed to vary stimulation parameters including pulse amplitude in a range from 0 mA to 10 mA, pulse width in a range from 50 μs to 500 μs, pulse frequency in a range from 5 Hz to 250 Hz, stimulation modes (e.g., continuous or cycling), and electrode configuration (e.g., anode, cathode, or off), to achieve the optimal therapeutic outcome specific to the patient. This programming allows for an optimal setting to be determined for each patient even though each parameter may vary from person to person.

As shown in FIGS. 5A and 5B, the IPG 10 may include a header portion 11 at one end. The header portion 11 houses a feed-through assembly 12, connector stack 13, and a communication antennae 16 to facilitate wireless communication with the clinician programmer and/or the patient remote. The IPG 10 includes a case 17, which houses the circuitry 23 including a printed circuit board, memory and controller components that facilitate the electrical pulse programs described above. A battery 24 is also contained within the case 17. In some embodiments, the battery is a primary cell battery that is non-rechargeable. In some embodiments, the battery is a lithium manganese dioxide (Li—MnO₂) battery (e.g. Litronik LiS 3150 MK battery having a capacity of 1200 mAh and a nominal voltage of 3.1 V). However, it is appreciated that any suitable battery may be used as needed for a particular application.

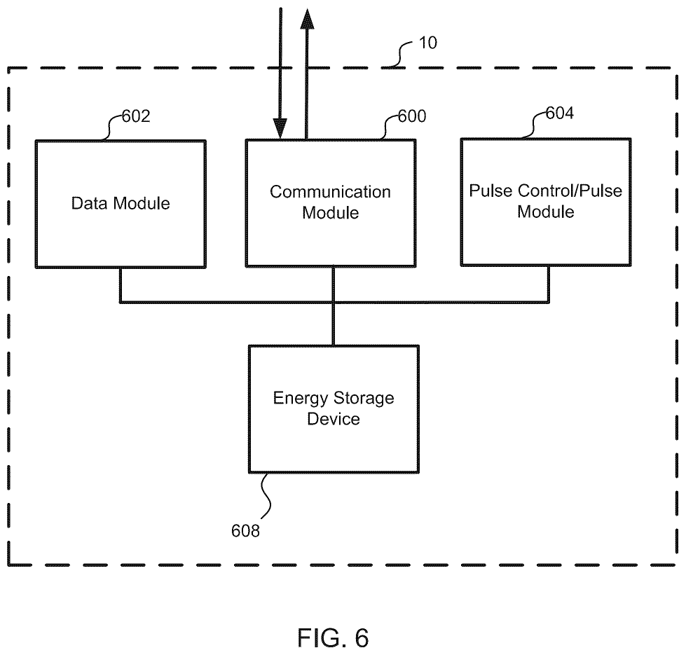

FIG. 6 shows a schematic illustration of one embodiment of the architecture of the IPG In some embodiments, each of the components of the architecture of the IPG 10 may be implemented using the processor, memory, and/or other hardware component of the IPG 10. In some embodiments, the components of the architecture of the IPG 10 may include software that interacts with the hardware of the IPG 10 to achieve a desired outcome, and the components of the architecture of the IPG 10 may be located within the housing.

In some embodiments, the IPG 10 may include, for example, a communication module 600. The communication module 600 may be configured to send data to and receive data from other components and/or devices of the exemplary nerve stimulation system including, for example, the clinician programmer 60 and/or the patient remote 70. In some embodiments, the communication module 600 may include one or several antennas and software configured to control the one or several antennas to send information to and receive information from one or several of the other components of the IPG 10.

The IPG 10 may further include a data module 602. The data module 602 may be configured to manage data relating to the identity and properties of the IPG 10. In some embodiments, the data module 602 may include one or several databases that may, for example, include information relating to the IPG 10 stored on a memory device. This information may include, for example, the identification of the IPG 10 or one or several properties of the IPG 10. In one embodiment, the information associated with the property of the IPG 10 may include, for example, data identifying the function of the IPG 10, historical stimulation program data, power consumption of the IPG 10, and battery consumption data, which can include battery usage data of one or more types, cumulative battery consumption value or values, data identifying the charge capacity of the IPG 10 and/or power storage capacity of the IPG 10, and various monitored parameters, including battery voltage measurements.

The IPG 10 may include a pulse control 604. In some embodiments, the pulse control 604 may be configured to control the generation of one or several pulses by the IPG 10. In some embodiments, for example, this may be performed based on information that identifies one or several pulse patterns, programs, or the like. This information may further specify, for example, the frequency of pulses generated by the IPG 10, the duration of pulses generated by the IPG 10, the strength and/or magnitude of pulses generated by the IPG 10, or any other details relating to the creation of one or several pulses by the IPG 10. In some embodiments, this information may specify aspects of a pulse pattern and/or pulse program, such as, for example, the duration of the pulse pattern and/or pulse program. In some embodiments, information relating to and/or for controlling the pulse generation of the IPG 10 may be stored within the memory.

In some embodiments, the pulse module 604 may include stimulation circuitry. The stimulation circuitry may be configured to generate and deliver one or several stimulation pulses, and specifically may be configured to generate a voltage driving a current forming one or several stimulation pulses. This circuitry may include one or several different components that may be controlled to generate the one or several stimulation pulses, to control the one or several stimulation pulses, and/or to deliver the one or several stimulation pulses.

The IPG 10 includes an energy storage device 608, such as a battery. In the embodiments described herein, the IPG 10 is powered by a primary cell battery (e.g. non-rechargeable battery). Over the life of the battery, the battery capacity and voltage depletes due to various types of use (e.g., delivery of stimulation pulse, self-discharge, energy consumption during a communication with external device, etc.). As the battery capacity depletes, the amount of energy available for functioning of the IPG 10 decreases over time. The battery capacity depletion may vary depending on the therapy parameters, patient-specific use, and various other factors. The battery capacity depletion is also influenced by multiple factors, which can include stimulation frequency, lower battery voltage, communication frequency, etc.) that may variably affect or accelerate the battery capacity depletion, while other factors (e.g., self-discharge, standard RF communication polling) may cause battery capacity depletion at approximately a constant rate. Battery life can vary substantially due to the various usage factors noted above.

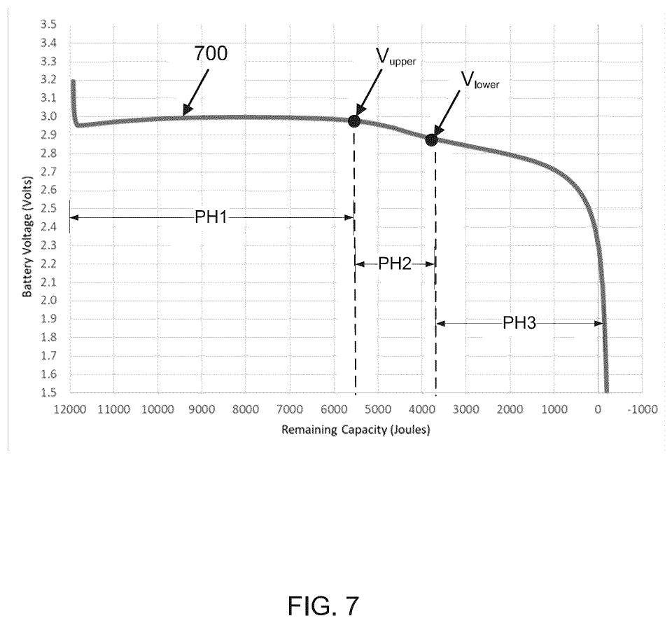

Many systems rely on voltage monitoring to determine estimates based on a standard battery discharge curve or a lookup table. However, as may be seen in FIG. 7, the voltage plot 700 of the discharge curve of a battery may be non-linear and highly variable. As shown, there may be an initial drop, then a steady increase for much of the life of the battery, after which the voltage begins to steadily decline and then exponentially drop towards the end of the life of the battery. Accordingly, the voltage-based approach of determining battery capacity is relatively inaccurate for most of the life of the battery, and once the voltage drops significantly, there remains only a limited time remaining before the end of life occurs and the battery must be replaced. While other approaches have sought to determine battery capacity by estimating battery discharge and subtracting these discharge estimates from battery capacity at the beginning of use, these approaches inherently include errors. Over time, these errors cause the estimate of battery capacity to become increasingly inaccurate, which can lead to premature battery replacement or failure to provide sufficient notice of the end of battery life. Moreover, due to the fundamental differences between these various approaches, the battery capacity estimates from these approaches may be quite different, such that the remaining battery capacity estimate cannot reasonably be relied upon by the clinician or patient. Thus, there remains a need for improved methods and systems that provide the patient and clinician with more accurate reliable estimates for remaining battery capacity. Based on remaining battery capacity estimate, the patient and clinician may be notified if a battery unexpectedly depletes faster than anticipated so that IPG may be replaced at appropriate time. However, determining a remaining battery capacity is not a trivial task.

As discussed herein, a remaining battery capacity may be a function of a de-rated battery capacity provided by a manufacturer, system (e.g., stimulation circuitry including hardware and software of IPG 10) design parameters, patient impedance and programmed stimulation parameters (e.g., current, pulse width, frequency, ramp duration, cycling, number of cathodes, etc.), or other parameters as discussed herein.

FIG. 7 illustrates an example of a battery discharge curve 700 that shows the battery voltage as a function of the battery capacity over the useful life (i.e., period of use) of a battery. The curve shown is for a Li—MnO₂ primary cell battery. It is appreciated that various other types of batteries include similar discharge curves, particularly Lithium-based primary cell batteries. In this discharge curve, after an initial period where the voltage remains relatively constant (apart from an initial spike, drop and gradual rise), the battery voltage begins to steadily decrease as the battery capacity decreases. The overall rate at which battery depletion occurs may vary depending on the extent of usage over the period of time. Since the voltage remains relatively steady and includes a gradual rise during this initial period, which is typically between four and six years, monitoring of voltage during this period to determine remaining battery capacity is generally not accurate. However, by utilizing an approach that accounts for the various types of battery usages that occur during this time period relative to a battery capacity at beginning of use can provide a relatively accurate estimate of remaining battery capacity. Once the voltage steadily declines as the battery approaches end of life, monitoring of the voltage becomes a better indicator as to remaining battery capacity because the battery voltage can correspond to a characteristic discharge curve for the respective battery. For this battery, this steady decline typically lasts about 1 to 2 years, following by a steeper decline and drop in the latter phase, which lasts about three years. However, during the interim time period between this initial phase and the latter phase, neither the first consumption-based method nor the second voltage-based approach is entirely accurate. Moreover, given the fundamental differences between these approaches, each can yield considerably different estimates such that neither can reasonably be relied upon.

In order to overcome the above dilemmas and provide an estimation of remaining battery capacity that is more reliable and consistent over the entire life of the battery, the methods and systems herein utilize a method that divides the battery discharge into at least different phases, as shown in FIG. 7. These differing phases include an initial phase PH1, an intermediate phase PH2, and a tertiary phase PH3.

During the initial phase PH1, the curve 700 may be relatively flat at or near the nominal voltage. After an initial spike and drop, the voltage remains relatively stable and only exhibits a slight rise during this period. Once the voltage crosses an upper battery voltage threshold Vᵤₚₚₑᵣ the voltage begins to steadily decline. In some embodiments, Vᵤₚₚₑᵣ corresponds to about 95%-99% of the nominal battery voltage at full battery capacity, typically about 96% (e.g. 2.974 V for a battery having a nominal voltage of 3.1 V). In the example shown, the battery voltage at full capacity varies between 2.94 V and 3.3V for a battery and has a nominal voltage of 3.1 V such that Vᵤₚₚₑᵣ is about 2.974 V. While voltage remains above Vᵤₚₚₑᵣ, for example during initial phase PH1, the remaining battery capacity may be estimated to be greater than a lower batter capacity BC_{lower} threshold (e.g., 45% of full capacity). In some embodiments, during the initial phase PH1, an accurate prediction of battery capacity may be made based on energy consumption data associated with energy used by various loads on the battery over the elapsed period of use. At voltages below the upper battery voltage Vᵤₚₚₑᵣ threshold, it was observed that the remaining battery capacity can increasingly be estimated as a function of the battery voltage. Accordingly, the method utilizes equations to predict the remaining battery capacity below this Vᵤₚₚₑᵣ threshold that may be derived, at least partly, as a function of the battery voltage.

During the intermediate phase PH2, which occurs between the Vᵤₚₚₑᵣ threshold and a lower battery voltage V_{lower} threshold, the voltage steadily declines such that the remaining battery capacity may be determined as a linear function of the voltage. The V_{lower} threshold may be a voltage between 85%-95% of the battery voltage at full battery capacity (e.g. about 93%, 2.87 V for a battery having a nominal voltage of 3.1 V). As noted above, however, the estimate determined based on battery consumption at the Vᵤₚₚₑᵣ threshold may be inconsistent from an estimate based on the voltage at Vᵤₚₚₑᵣ threshold. Therefore, in some embodiments, in order to provide a more consistent estimate of remaining battery capacity, the method includes equations that blend a first estimate based on battery consumption with a second estimate based on voltage. These estimates may be blended linearly with the first estimate fully weighted at the Vᵤₚₚₑᵣ threshold and the second estimate fully weighted at the V_{lower} threshold.

During the tertiary phase PH3, where the voltage is below the V_{lower} threshold, the remaining battery capacity may be predicted based only on the voltage, without any battery consumption data. In some embodiments, the determination uses a polynomial function of the voltage. In some embodiments, the polynomial function can approximate curves that follow the battery characterization data of a given battery type.

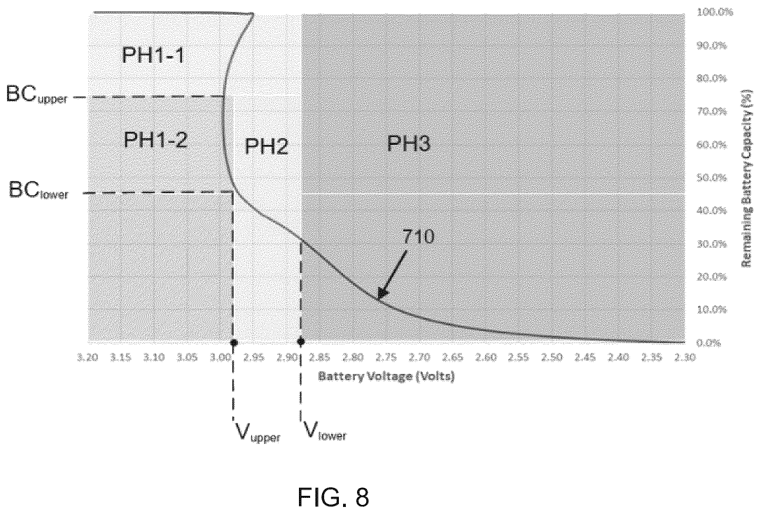

FIG. 8 shows the battery discharge curve as a voltage plot 710 with the axes reversed to show remaining battery capacity (e.g., as a percentage of full capacity) as a function of the voltage. FIG. 8, shows various distinct phases and sub-phases of the curve that may be used in the method for estimating remaining battery capacity. Additional details, parameters, and equations of the method are discussed in further detail below.

During the initial phase PH1, shown in FIG. 8, the initial spike and drop is followed by a slight voltage rise in voltage which can complicate the estimation since the same voltage appears at different times. Accordingly, the initial phase PH1 may be divided into a first subphase PH1-1 and a second subphase PH1-2. The first subphase PH1-1 differentiates between lower battery voltages seen early in the life of the battery and those same lower battery voltages seen later in the life of the battery. Accordingly, a remaining battery capacity may be estimated using different equations for the first subphase PH1-1 and the second subphase PH1-2. The following discussion provides an overview of a process of estimating a remaining battery capacity for different phases observed over the period of use of the implantable device (e.g., IPG).

In one aspect, in the initial phase of battery usage, a first consumption-based method (e.g., a dead reckoning) that tracks or accounts for battery energy consumed over an elapsed period of use may be used to estimate a remaining battery capacity. For example, a remaining battery capacity may be estimated by computing a total battery energy consumption associated with energy use by different IPG electrical loads and subtracting this from the battery capacity of a new battery (e.g., a de-rated capacity provided by a manufacturer). In later phases, a second method based, at least partly, on the voltage of the battery may be used to estimate the remaining battery consumption.

The first consumption-based method (e.g., a dead reckoning) can make accurate predictions of remaining battery capacity when an actual battery energy consumption for different usages (e.g., self-discharge, communication with external devices, stimulation pulse delivery, etc.) is tracked accurately. However, the first method may over-estimate past battery usage as the battery energy consumption for different usages may be based on conservative assumptions. Therefore, when the voltage drops below the Vᵤₚₚₑᵣ threshold (e.g., 2.974 V) and the estimated remaining battery capacity is determined to be below a lower battery capacity BC_{lower} threshold (e.g., between 40 to 50%, about 46%), the first method is likely predicting an overly conservative (lower as compared to actual) remaining battery capacity. To avoid discontinuity in the remaining battery capacity estimate, when the battery voltage first reaches Vᵤₚₚₑᵣ (e.g., 2.974 V) and/or is at or below an upper battery capacity BC u pp e r threshold (e.g. 70-80%, 75%), the estimation produced by the first consumption-based approach may have a lower limit of a lower battery capacity BC_{lower} threshold (e.g. between 40-50%, about 46%). When the voltage then drops below Vᵤₚₚₑᵣ, if dead reckoning predicts a remaining capacity below the lower limit, going forward the remaining capacity value is calculated by the second voltage-based method. The second method may estimate the remaining battery capacity as a function, at least partly, of battery voltage, instead of the battery energy consumed by the IPG.

The process of estimating the remaining battery capacity can also consider a scenario in which the first method may be overly optimistic. In that case, the remaining battery capacity may be determined by combination or blending of results from the first method and the second method. In some embodiments, this blending or combination is applied during the intermediate phase PH2. In FIG. 8, the intermediate phase PH2 is a transition region characterized by battery voltages between the Vᵤₚₚₑᵣ threshold and V_{lower} thresholds (e.g., between 2.974 and 2.870 V). In some embodiments, the blending may be such that the remaining battery capacity determined by the first method dominates near the V u pp e r threshold (e.g., 2.974 V) while the remaining capacity determined by the second method dominates near the V_{lower} threshold (e.g., 2.870 V).

In some embodiments, an unexpected rate of battery depletion may occur. For example, a failure of the power system (battery, circuit, etc.) inside the IPG may cause an unexpectedly faster rate of battery depletion. Such a situation will lead to the battery voltage being lower much earlier than estimated based on the first method or the blending of the first and second methods. As such, in the tertiary phase PH3, characterized by the battery voltage depleting below the V_{lower} threshold (e.g., 2.870 V), the remaining capacity may be based only upon a measured battery voltage. In other words, the remaining battery capacity calculated using the first method (e.g., the dead reckoning method) may be ignored in this phase. Thus, a battery unexpectedly depleting faster than anticipated will be indicated as such to the patient, irrespective of the calculation based on usage of the implantable device.

Set forth below is a description of various operations and methods that use battery power and cause the battery to be depleted. All or some of these usages of battery power may be considered in determining the estimated remaining battery capacity by battery depletion data. Such usages can include the following:

In some embodiments, the fixed battery energy usage data set 921 includes a first energy consumption data set associated with a battery energy consumed or lost due to battery self-discharge (Usage 1). The battery self-discharge may be an amount of energy consumed internally in the battery. Because the self-discharge occurs inside the battery, the first usage may not be measurable external to the battery. In some embodiments, parameters used for determining the first energy consumption data set may include, but are not limited to, an annual self-discharge rate (e.g., in percentage), battery de-rated capacity (e.g., in joules), or other related factors. For example, the battery self-discharge may be computed in joules per day as a function of the de-rated capacity, and the self-discharge rate.

In some embodiments, the fixed battery energy usage data set 921 includes a second energy consumption data set associated with battery energy consumed by a communication polling at a first scan rate to determine whether the external device is requesting to communicate (Usage 2). In some embodiments, the second energy consumption data set comprises an amount of battery energy consumed during a periodic scan that the IPG conducts to determine if the external device (e.g., the remote 70 or the clinician programmer 60) is requesting to communicate with the IPG. The energy consumption over time depends on several factors and may be difficult to model analytically. Therefore, a parameter value from an actual measurement may be used to determine the battery energy consumption. Parameters used for determining the second energy consumption data set may include, but are not limited to, energy used from the battery for receiving radio frequency (RF) signals (e.g., in μJoules per scan), an energy margin provision for potential future increase in energy needed per scan (e.g., in μJoules per scan), a modeled energy extracted from the battery for receiving an RF signal (μJoules per scan), an RF scan interval (e.g., seconds), a number of RF scans per day, or other related parameters. Accordingly, the second energy consumption data set may include a total polling energy computed as a function of the aforementioned parameters.

In some embodiments, the fixed battery energy usage data set 921 includes a third energy consumption data set associated with battery energy consumed by a radio frequency communication involving a communication with the external device (Usage 3). In some embodiments, the third energy consumption data set comprises an amount of energy consumed when the IPG (e.g., 10) has a communication episode with the external device (e.g., the remote 70 or the clinician programmer 70). The energy consumption over time depends on several factors and may be difficult to model analytically. Therefore, values from an actual measurement during different stages of the communication may be used in determining model parameters of an analytical model or an empirical model to calculate an aggregate value of energy consumption. For example, parameters used for determining the third energy consumption data set may include, but are not limited to, an energy (e.g., measured in mJoules) consumed for a predetermined amount (e.g., in seconds) of a conversation, an energy margin (e.g., assumed in mJoules) provided for future changes, a number of conversations per episode, number of episodes per year, pro-rata number of scans per day or other factors. Accordingly, the third energy consumption data set may include a total prorated energy (e.g., in Joules per day) may be computed as a function of the aforementioned parameters.

In some embodiments, the fixed battery energy usage data set 921 includes a fourth energy consumption data set associated with battery energy consumed by a communication at a second scan rate to determine whether the external device is requesting to communicate (Usage 4). In some embodiments, the implantable device may be configured to poll for a follow-up conversation with the external device at a second scan rate. For example, after an RF conversation session, for the following predetermined time period (e.g., 60 seconds), the IPG may poll for a follow-up conversation with a patient remote or the clinician programmer at a faster rate (e.g., period 4.6 seconds) compared to a normal polling rate (e.g., period of 16 seconds). Such functionality may be intended to make the IPG more responsive for immediate follow-up conversations. A model for such communication may be developed using the parameters related to the energy usage from RF polling in the second energy consumption data set. Additionally, parameters used for determining the fourth energy consumption data set may include, but are not limited to, a second scan interval (e.g., seconds), a duration of persistence after interaction with the external device (e.g., in mins), a number of scans per episode, an energy consumed from battery to receive an RF signal (e.g., in μJoules per scan), an energy consumed from battery per episode for receiving an RF signal (e.g., in mJoules), an energy per episode for memory write (e.g., in mJoules), a number of episodes per year, a prorated number of episodes per day, or other parameters. Accordingly, the fourth energy consumption data set may include a total prorated RF communication polling energy in Joules per day computed as a function of the aforementioned parameters.

In some embodiments, the fixed battery energy usage data set 921 includes a fifth energy consumption data set associated with battery energy consumed by housekeeping tasks performed by software within the implantable device (Usage 5). In some embodiments, the fifth energy consumption data set comprises an energy consumption for the myriad housekeeping functions the IPG software performs periodically. Such energy consumption may be modeled. The value used in the model may be determined from measurement data. Adequate margin over the measured value may be modeled to allow for future software versions possibly adding additional housekeeping tasks and thus using more energy. Parameters used for determining the fifth energy consumption data may include, but are not limited to, an energy consumed from the battery for housekeeping tasks (e.g., in Joules per day), an energy margin to allow for possible increase in energy usage in the future as software changes, or other parameters. Accordingly, the fifth energy consumption data may be a total energy in Joules per day computed as a function of the aforementioned parameters.

In some embodiments, the fixed battery energy usage data set 921 includes a sixth energy consumption data set associated with battery energy consumed by a quiescent current present in the implantable device (Usage 6). For example, the sixth energy consumption data set includes an amount of energy used from the battery to provide a quiescent current that the IPG consumes all the time after the battery has been connected to a circuit board of the IPG. Parameters used for determining the sixth energy consumption data set may include, but are not limited to, system quiescent current extracted from the battery (e.g., in μA), a voltage at which quiescent current discharge occurs (e.g., in V), or other parameters. Accordingly, the sixth energy consumption data set may be a total energy in Joules per day computed as a function of the aforementioned parameters. In some embodiments, a total daily energy consumed from the battery (e.g., in Joules per day) may be computed as a sum of the first to sixth energy consumption data set.

In some embodiments, the fixed battery energy usage data set 921 includes a seventh energy consumption data set associated with the energy discharged or lost from the battery due to on-the-shelf-discharge experienced by the battery before connecting to the implantable device such as the IPG 10 (Usage 7). In some embodiments, the period of self-discharge of the battery of an IPG may be divided into following stages: (i) self-discharge of the battery that occurs from the moment it is manufactured; (ii) once the battery is attached, the IPG circuit experiences the daily energy consumption (e.g., the first to sixth energy consumption data set); (iii) after assembly, the IPG undergoes tests at the IPG assembler; and (iv) after sterilization, the packaged IPG is tested at another manufacturer location. The duration for various stages during the shelf life may be estimated. Parameters used for determining the seventh energy consumption data may include, but are not limited to, a first duration characterized by a time period from battery manufacturer to attachment to the IPG circuit (e.g., in months), a second duration characterized by a time period from battery attachment to the IPG assembly (e.g., months), a third duration characterized by a time period from the IPG assembly to sterilization and ready to ship (e.g., in months), and a fourth duration characterized by a time period from ready to ship to implant (e.g., months). Accordingly, a total duration from battery manufacture to implant (e.g., in months) may be computed as a sum of the first duration to the fourth duration. Similarly, a duration from battery attachment to circuit to implant may be computed as a sum of the second duration to the fourth duration. Based on the durations (e.g., the first to fourth durations) and the battery self-discharge parameter (e.g., in Joules determined during the first energy consumption data set), an amount of energy consumed from the battery during the different durations may be determined.

In addition to battery energy consumed during the first to fourth durations, an energy margin (e.g., in Joules) to allow increase in future test fixture upgrades may be included. Furthermore, an energy (e.g., in Joules) used during packaged IPG testing at the IPG manufacturer may be included. Based on the aforementioned energy computations, a total energy used while on shelf may be computed as sum of energy consumed in the first to fourth durations, energy margins, or other parameters. These aforementioned energy values associated with battery discharge while on shelf may be included in the seventh energy consumption data set.

In some embodiments, the fixed battery energy usage data set 921 includes an eighth energy consumption data set associated with battery energy consumed due to communications with the external device during implanting of the implantable device (Usage 8). For example, the IPG 10 communicates with the clinician programmer 70 during the time of implant. After implantation there is usually a follow up visit with a clinician. It may be also anticipated that after an IPG is implanted, the patient may come into the clinician's office a couple of times, such as, for a mid-life checkup and for a check towards the IPG's end of life. The amount energy used by the IPG when communicating with the clinician programmer, is similar to the amount of energy used by the IPG when communicating with the patient remote. The energy usage for one communication session between the IPG and the patient remote may be modeled as a predetermined period (e.g., 30 second) of the communication session.

Parameters used for determining the eighth energy consumption data may include, but are not limited to: (i) a total energy consumed for one conversation (e.g., in mJoules) with a first external device (e.g., the patient remote) for a predetermined time period (e.g., 30 seconds), (ii) a number of first external device (e.g., patient remote) equivalent conversations with a second external device (e.g., clinician programmer) during a second time period (e.g., 45 minutes) of the implant procedure, (iii) a number of first external device (e.g., patient remote) equivalent conversations with a second external device (e.g., clinician programmer) during a third time period (e.g., 10 minutes) of follow up procedure, (iv) a number of first external device (e.g., patient remote) equivalent conversations with a second external device (e.g., clinician programmer) during a IPG mid-life follow up (e.g., 10 minutes), (v) a number of first external device (e.g., patient remote) equivalent conversations with a second external device (e.g., clinician programmer) during a IPG end-of-life follow up (e.g., 10 minutes), and/or (vi) a number of first external device (e.g., patient remote) equivalent conversations with a second external device (e.g., clinician programmer) during the IPG lifetime period of use. The aforementioned parameters (i)-(vi) and the energy consumption related to these parameters discussed herein may be used to estimate the energy consumption for the eight energy consumption data set. Thus, the fixed energy consumption data set 921 may include several different types of energy consumption data and related parameters.

Similarly, the active battery energy consumption data set 922 includes data associated with parameters affecting an amount of energy required to deliver a stimulation pulse. The amount of energy consumed from the battery depends on stimulation pulse delivery related parameters (e.g., stimulation pulse frequency, current amplitude, etc.), hardware and software components, patient tissue characteristics, or other parameters. In some embodiments, energy associated with stimulation pulse delivery may be determined using a system model of the implantable device (e.g., IPG 10).

In some embodiments, for determining energy consumption associated with stimulation pulse delivery, a model for the IPG hardware and software may be developed. For example, the energy model may be a function of an energy consumption by a central processing unit (CPU), on which the IPG software resides, and another energy consumption by analog circuits used to generate a stimulation pulse. The CPU energy consumption may be characterized by a ninth energy consumption data set, and the hardware energy consumption may be characterized by a tenth energy consumption data set, both of which are further discussed in detail below.

In some embodiments, the ninth energy consumption data set includes parameters related to battery energy consumed by the processor, controller or central processing unit (CPU) for release or activation of a stimulation pulse (Usage 9). The parameters related to the CPU energy consumption may have a weak relationship to a duration of the pulse. Also, the CPU energy consumption may not depend on the energy used in delivering the stimulation pulse. Therefore, the CPU energy consumption determined may be considered as a fixed amount of energy used for each stimulation pulse.

The parameters used for determining the ninth consumption data may include, but are not limited to, CPU start up time (μSec) and CPU start up current (mA). The energy consumption for start-up of each stimulation pulse (μJoules per pulse) may be computed as a function of the CPU start up time and current. Additionally, the parameters may include, but are not limited to, CPU Processing Duration (μSec) and CPU Processing Current (mA). The energy consumption during CPU processing (μJoules per pulse) may be computed as the processing duration and current.

In some embodiments, the stimulation pulse may be delivered in phases. In this case, the parameters may additionally include, but are not limited to, a first phase duration assumed (μSec) and CPU current during the first phase (mA) of delivery of the stimulation pulse. The energy consumed during the first phase (μJoules per pulse) delivery of stimulation pulse may be computed as a function of the first phase duration and current. Furthermore, the parameters may include, but are not limited to, an interphase delay (μSec), a second phase duration assumed (μSec), CPU current during the interphase delay, and the second phase delivery current (mA). The energy consumed during the interphase delay and the second phase (μJoules per pulse) may be computed as a function of the aforementioned parameters. Accordingly, the aforementioned parameters and the CPU energy consumption related to these parameters may be used to estimate the CPU energy consumption. As such, the ninth consumption data set may include parameter values related to start up, processing, different phases, and corresponding energy consumptions. A total CPU energy consumption may also be computed as a sum of the individual energy consumptions during start up, processing, and different phases (μJoules per pulse) and included in the ninth energy consumption data set.

In some embodiments, the tenth energy consumption data set includes parameters related to battery energy consumed by the analog circuits of the IPG to deliver a stimulation pulse (Usage 10). The parameters used for determining the tenth energy consumption data set may include, but are not limited to, a specified stimulation current amplitude, a specified stimulation pulse width, a specified stimulation frequency (e.g., at 14 Hz), a patient's resistive portion (e.g., tissue resistance), a specified duty cycle, and a number of pulses per day.

The tenth energy consumption data set parameters may include a maximum possible current (mA) used for the stimulation pulse delivery. The voltage across a patient may be limited to a particular voltage (e.g., 9 volts). A double layer capacitor at the electrode/tissue interface (e.g., 0.47 μF) can build up a voltage during the first phase of the stimulation pulse. Therefore, the maximum current that is deliverable to the patient can depend upon the patient impedance and pulse duration. Accordingly, a maximum possible current may be computed as a function of the particular voltage, patient impedance and resistance, and the pulse duration.

The tenth energy consumption data set parameters may include a limited stimulation current amplitude (μA) used for stimulation pulse delivery, which may be a lower bound of a programmed current and the maximum possible current calculated above. The tenth energy consumption data set parameters may include a stimulation voltage (mV) at the patient's resistive portion, which may be a voltage at the start of the first phase, assuming the patient's resistive portion is purely resistive. The tenth energy consumption data set parameters may include a voltage buildup on a patient's portion thereby serving as a capacitor during delivery of the first phase of the stimulation pulse. The tenth energy consumption data set parameters may include a voltage drop due to switches, sense resistor, and system circuit impedance. The voltage may drop internally in the IPG due to internal components impedances, depending upon current. The tenth energy consumption data set parameters may include a total stimulation voltage required from the stimulation power supply and may be calculated as a sum of the stimulation voltage, voltage buildup, and voltage drop.

In some embodiments, the hardware components of the IPG may include a power converter such as a buck (e.g., steps down a voltage) and/or a boost (e.g., steps up a voltage) power supply. Accordingly, the tenth energy consumption data set parameters may include a minimum buck and a maximum boost specification. The voltage used to deliver the stimulation pulse may be bounded. The lower bound may correspond to a minimum voltage that a buck power supply can deliver. The upper bound may correspond to a maximum voltage that a boost power supply can deliver. In some embodiments, depending upon a voltage demand during the stimulation pulse delivery, either the buck or the boost power supply will be engaged. Furthermore, the tenth energy consumption data set parameters may include a power convertor efficiency, which may depend upon the buck and/or the boost. The tenth energy consumption data set parameters may include an amount of energy delivered by a stimulation power supply during the first phase. The energy delivered may be computed as a function of a stimulation current, a stimulation voltage, and duration of the first phase. The tenth energy consumption data set parameters may include an energy consumed (μJoules) from battery per pulse based on the efficiency of the buck and/or the boost converter.

The tenth energy consumption data set parameters may include a quiescent current (μA) consumed by the buck and/or the boost convertor. For example, for the buck convertor, a quiescent current lasts for an entire duration of the stimulation pulse. While, for the boost convertor, a quiescent current lasts for the first phase duration only. Further, the parameters may include a quiescent current energy per pulse (μJoules), which may be computed as a function of the quiescent current(s) for the converter, the duration of the quiescent current(s), and the stimulation frequency. The tenth energy consumption data set parameters may include a total analog circuit energy, which may be computed as a sum of the energy consumed (μJoules) from battery per pulse and the quiescent convertor energy per pulse (μJoules). Thus, the active battery energy consumption data set 922 can include a total energy related to stimulation pulse delivery, which may be computed as the sums energy consumptions related to the digital (e.g., CPU) and the analog circuits for delivery of each stimulation pulse.

In some embodiments, based on the battery energy consumption data set and related parameters, a longevity or expected life (e.g., in days) of the battery may be computed. In one aspect, the battery longevity may be calculated by the following equation:

$$Longevity ( Days ) = Usable Battery Capacity / Usage Per Day \\ Usable Battery Capacity = Derated Battery Capacity - ( Usage 7 + Usage 8 ) \\ Usage Per Day = ( Usage 1 thru Usage 6 ) + ( E * F * C ) \\ E = Energy Factory = ( Usage 9 + Usage 10 ) * ( Number of pulses at 14 Hz per day ) \\ F = Frequency Factor = Stim Frequency / 14 \\ \begin{matrix}C = Cycling Factor ( C = 1 , if no cycling is used ) \\ = \frac{( Cycling\_On \_Time \_Secs + 5 * Ramp\_Time \_Secs )}{( Cycling\_On \_Time \_Secs + Cycling\_Off \_Time \_Secs )}\end{matrix}$$

As indicated above, the longevity of the battery may be a ratio of a usable battery capacity and energy consumption per day. The usable battery capacity may be computed as a difference between the de-rated battery capacity and a sum of the total energies computed in the seventh and eighth energy consumption data set. The energy consumption per day may be computed as a function of the total energies in the first through sixth energy consumption data set, an energy factor (E), a frequency factor (F), and a cycling factor (C). For example, the energy factor (E) may be a function of the total energies in the ninth and tenth energy consumption data set, and a number of pulses at a given frequency (e.g., 14 Hz per day). The frequency factor (F) may be a function of a stimulation frequency and a given frequency (e.g., 14 Hz). The frequency factor (F) accounts for energy impact from use of a stimulation frequency other than the given frequency (e.g., 14 Hz). The cycling factor (C) accounts for cycling through starting, stopping, and ramping stimulation that uses extra energy. This may be accounted for by multiplying a ramp time in calculating the cycling factor (C).

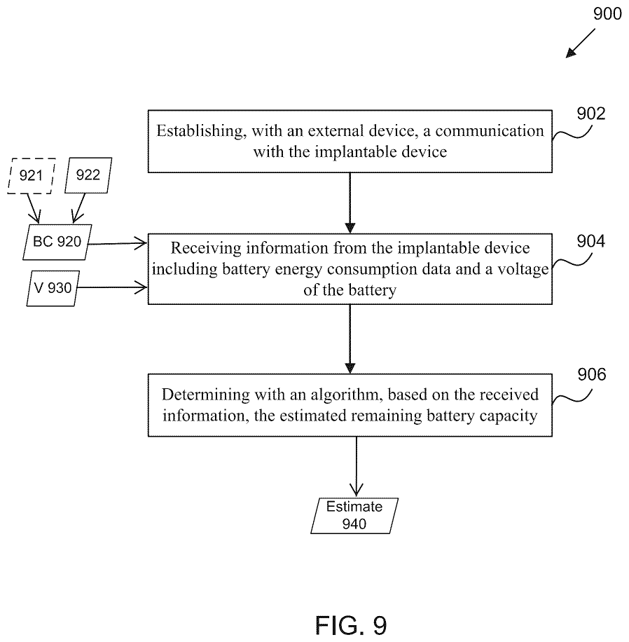

FIG. 9 is an example flow chart of a method 900 for determining an estimated remaining battery capacity of a battery in an implantable device over a cycle of use. The method 800 includes estimating the remaining battery capacity based on battery energy consumption and battery voltage during different phases of the battery life. In some embodiments, the battery may be a non-rechargeable primary cell battery of the IPG 10. For example, the battery may be a lithium manganese dioxide (Li—MnO₂) battery. Accordingly, the method 800 may involve collecting and analyzing data related to battery energy usage associated with the IPG, measuring battery voltage from the IPG and using the data to estimate the remaining battery capacity.

Most of the battery energy consumed by the IPG may be used after power conversion to a fixed voltage. In other words, most of the energy consumed may be independent of the battery voltage. As the remaining capacity of the battery is depleted, the battery voltage declines and dips (e.g., as discussed with respect to FIGS. 7-8). For a given amount of energy, more current is drawn from the battery as it is depleted. Therefore, the ability of the battery to deliver energy (e.g., in Joules) may be preferred over its ability to source current (amps). Accordingly, the method 800 may determine battery capacity in terms of energy (e.g., in Joules) even if the battery manufacturer has rated the battery in terms of its current delivery capability (mAh). Although, the method determines capacity in terms of energy (e.g., in Joules), the method may also apply and be converted to measure capacity in terms of watt-hours, milliamp hours, coloumbs or percentage of rated or maximum capacity. The method 900 is discussed in further detail below.

In example method 900, step 902 involves establishing a communication between the implantable device and an external device. In some embodiments, the implantable device may be an IPG for any neurostimulation therapy (e.g. brain, muscle, spinal, sacral, etc.). In some embodiments, the external device may be a clinician programmer, and/or a remote (e.g., the clinician programmer 60 and/or the patient remote 70 in FIG. 1), and/or any other electronic devices that may communicate with the IPG. The clinician programmer 60 may send or receive commands for operation of the IPG (e.g. for activating the IPG, delivering simulation pulse of certain frequency and energy) and communicate with the IPG to facilitate telemetry regarding performance or operational parameters of the IPG (e.g. receive battery consumption data, receive battery usage, historical stimulation program data/settings, etc.). The remote 70 may be configured to communicate and control parameters related to stimulation pulse delivery of the IPG. In some embodiments, a user (e.g., a patient) may change the stimulation parameters set by the clinician programmer 60 to deliver higher or lower stimulation pulse. As such, an actual battery usage may be higher or lower compared to an estimated battery usage of a program set by the clinician programmer 60.

Step 904 involves receiving information from the implantable device (e.g., the IPG 10) including battery energy consumption data BC 920 and a voltage V 930 of the battery. In some embodiments, the battery energy consumption data 920 includes data related to different energy consumptions of the battery that causes the battery capacity to deplete. In some embodiments, the battery energy consumption data includes a fixed battery energy usage data 921 related to a fixed amount of energy consumption from the battery for a particular purpose. In some embodiments, the battery energy consumption data set includes active battery energy consumption data 922 associated with stimulation pulse delivery. The fixed battery energy usage data set can include, but is not limited to, any of: battery energy consumption related to at least one of a fixed battery discharge, a periodic communication with the external device, housekeeping tasks related to functioning of software within the implantable device, or a quiescent current consumed by the implantable device, or any combination thereof.

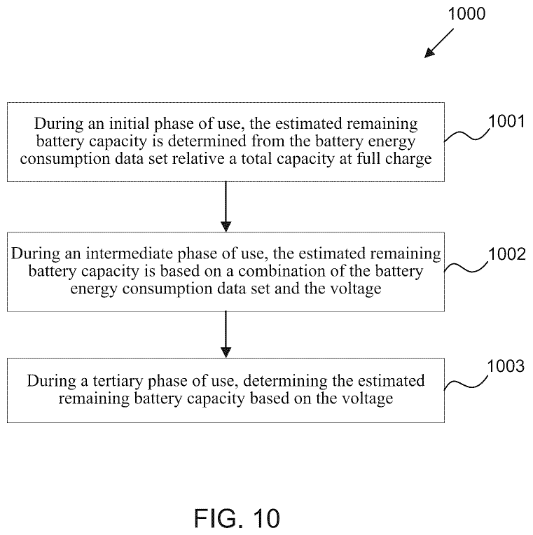

Step 906 involves determining, based on the received information, the estimated remaining battery capacity. In some embodiments, the estimated remaining battery capacity may be determined differently for different phases observed over a period of use. In some embodiments, at the step 906, the estimated remaining battery capacity is determined as shown in FIG. 10 and further discussed in detail below. After, which the external device outputs the battery capacity estimate 940 to the user, for example on the graphical user interface display.

In some embodiments, the communication is established at a patient visit with the clinician, at which the clinician programmer interrogates the IPG to determine the remaining battery capacity estimate (e.g. by direct communication between the IPG and clinician programmer). The estimate may be expressed in any suitable manner (e.g. as a % of total capacity, or as a remaining battery life of weeks/months/years/etc.). In some embodiments, the clinician programmer determines the battery capacity estimate at the session from the information obtained and indicates the estimate the estimate to the clinician, but does not otherwise store or maintain the estimate on the clinician programmer or patient profile. Since the estimate is not stored or maintained, the clinician programmer cannot update the estimate. In such embodiments, if a new or updated estimate is desired, the clinician programmer must initiate a new communication session and receive updated data and perform a new estimate according to the methods detailed herein. In some embodiments, the battery capacity estimate may be determined and/or displayed on a patient remote or other electronic device that may communicate with the IPG.

FIG. 10 shows an exemplary method 1000 of determining a remaining battery capacity estimate in accordance with some embodiments. The method includes steps of 1001, 1002, and 1003 that correspond to differing phases (e.g., an initial phase, an intermediate phase, and a tertiary phase) over the period of use over the lifetime of the battery. In some embodiments, each of the steps 1001, 1002, 1003 are performed at differing times, rather than simultaneously or in a sequence. In some embodiments, the step 1001, 1002, 1003 may be selectively performed based on trigger conditions defined as a function of the battery voltage and/or the remaining battery capacity, according to the voltage and capacity thresholds described herein. Step 1001 is performed during an initial phase of use, in which the estimated remaining battery capacity is determined from the battery energy consumption data set relative to a battery capacity at full charge when the battery is first put into service. This full charge value of capacity may be derated in order to provide a more conservative approach to estimating battery capacity to ensure adequate notice of time for battery replacement. Step 1002 is performed during an intermediate phase of use, in which the estimated remaining battery capacity is based on a combination of the battery energy consumption data set and the voltage. Step 1003 is performed during a tertiary phase of use, in which the estimated remaining battery capacity is determined based on the voltage. When method 1000 is performed periodically over the usable life of the device (for example at clinician/patient visits conducted annually, or bi-annually), the differing steps will be performed at different times during the respective phases.

In one aspect, the voltages ranges assume a nominal battery voltage as well as an upper bounds of the battery voltage and a lower bounds of the battery voltage that indicates end of life. In the exemplary embodiment disclosed herein, for a battery having a nominal voltage of 3.1 V, an upper bounds of voltage is about 3.3 V and a lower bounds is about 2.3 V, which indicates end of life. The following represent example equations of the battery capacity estimation method described herein. It is appreciated that these equations are exemplary and that the recited thresholds are specific values that pertain to the exemplary battery described herein and that these concepts may be modified and applied to the parameters of various other batteries as needed.

In this initial phase (PH1 in FIG. 8), the estimated remaining battery capacity may be determined from the battery energy consumption data relative to a total capacity at full charge. For example, cumulative battery energy consumed by the IPG, as determined from the data, may be subtracted from the total capacity of the battery. The method using the battery energy consumption data set may be referred to as a first method or a dead reckoning method that involves tracking energy consumption for different usages. For example, the battery energy consumption data can include computing energy consumption for different types of usages e.g., the first energy consumption data set through the tenth energy consumption data set, as described previously.

In some embodiments, the initial phase may be divided into a first subphase (e.g., PH1-1 in FIG. 8) and a second subphase (e.g., PH1-2 in FIG. 8). In the first subphase (e.g., PH1-1), the first method may be applied to determine the remaining battery capacity. While in the second subphase (e.g., PH1-2), the remaining battery capacity may be calculated by the first method above, yet if overly pessimistic, the estimate may be assigned a particular value, for example a lower capacity threshold. For example, applying the first method in the second subphase may predict a lower remaining battery capacity than the actual remaining battery capacity. Hence, a floor may be applied to the remaining battery capacity during the second subphase.

In some embodiments, during the first subphase, the estimated remaining battery capacity is calculated by the first method when the capacity is greater than the upper battery capacity BCᵤₚₚₑᵣ threshold (e.g. 70-50%, about 75% of full capacity). In some embodiments, the first subphase may also be determined based on a further check of the voltage relative to a lower battery voltage V_{lower} threshold (e.g. 85-95%, about 92% of nominal battery voltage). In some embodiments, the V_{lower} threshold is 2.87 V for a battery having a nominal voltage of 3.1 V.

Referring to FIG. 8 as an example, when the voltage is greater than the V_{lower} threshold and the remaining battery capacity (e.g., determined by the first method) is greater than 75%, the battery may be considered to be in the first subphase PH1-1 and the estimated battery capacity may be considered an accurate prediction.

Referring back to FIG. 8, during the second subphase (e.g., PH1-2), a check may be performed to determine whether the voltage is greater than the upper battery voltage Vᵤₚₚₑᵣ threshold (e.g. 95-99% of nominal voltage, about 96%, 2.974 V for a battery having 3.1 V nominal voltage). In some embodiments, a check may be performed to determine whether the estimated remaining battery capacity (e.g., determined by the first method) is less than BCᵤₚₚₑᵣ threshold and greater than a lower battery capacity BC_{lower} threshold. For example, the BCᵤₚₚₑᵣ threshold may be between 70%-80% (e.g. 75%) of a full battery capacity, and the BC_{lower} threshold may be between than 40%-50% (e.g. 45%) of the full battery capacity. If these checks are satisfied, the remaining battery capacity may be determined by the first method may be considered to be an accurate prediction.

Referring to FIG. 8 as an example, during the second subphase PH1-2, when the voltage is greater than the Vᵤₚₚₑᵣ threshold (e.g., 2.974 V) and/or the estimated remaining battery capacity is below the BCᵤₚₚₑᵣ threshold (e.g. 75%), a value estimated by the first method may be used as the estimated remaining battery capacity. However, if the value estimated by the first method is less than the BC_{lower} threshold, the estimated remaining battery capacity determined by the first method may be considered too pessimistic and may be floored to the lower battery capacity BC_{lower} threshold, thereby discarding the estimated value by the first method.

During the intermediate phase of use (e.g., PH2 in FIG. 8), the estimated remaining battery capacity may be determined based on a combination of the battery energy consumption data 920 and the voltage 930. In some embodiments, during the intermediate phase, the battery energy consumption data 920 and the voltage 930 are combined linearly, non-linearly, weighted, or combined by various other methods. The present disclosure is not limited to a particular combination method.

In some embodiments, during the intermediate phase, a first estimate based on the first method utilizing battery energy consumption data and a second estimate based on a second method utilizing voltage are combined linearly such that battery energy consumption data set 920 is fully weighted upon commencement of the intermediate phase and the voltage is fully weighted at completion of the intermediate phase. In some embodiments, the first estimate may be the floored value from sub-phase PH1-2.

An example method to estimate the remaining battery capacity involves: (i) determining a remaining battery capacity as a first function ƒ1 of a battery voltage; and (ii) determining a remaining battery capacity based on the battery energy consumption data set 920, as discussed earlier. The first function ƒ1 may be an empirical equation based on measured and/or simulated data over a period of battery use. For example, the first function ƒ1 may be of a form a1*V_{b}+b1, where a1 and b1 are fitting coefficients and V_{b} is the battery voltage. The measured data and/or the simulated data comprises battery voltages, and a battery capacity measured/simulated for the period of use of the IPG. In some embodiments, the measured data may be collected from the IPG implanted in a patient. In some embodiments, the simulated data may be generated by using models associated with IPG. The simulation model of the IPG may include physics-based models of energy usage by IPG components (e.g., including hardware and software), that imitate a behavior of IPG in use, and other models configured to simulate IPG behavior and battery depletion.

Referring to FIG. 8 as an example, the intermediate phase PH2 corresponds to the battery voltage 930 between the upper and lower thresholds (e.g., between 2.974 V and 2.870 V). In some embodiments, if a remaining battery capacity (BC_{DR}) determined by the first method is less than a remaining battery capacity (BC_{BV}) determined based on the voltage, then the estimated remaining battery capacity 940 is assigned as BC_{BV}. Otherwise, the estimated remaining battery capacity 940 may be computed as a linear combination of a result (e.g., BC_{BV}) of applying the function ƒ1 (e.g., 1*V_{b} b1, where a1=1.4376082; b1=−3.8259548), and a result (e.g., BC_{DR}) of the first method using the battery energy consumption data. For example, a linear combination function may use a second function ƒ2 represented as a function of the voltage thresholds and the battery voltage V_{b} (i.e., 930) to combine the individual contributions from different methods. As an example, the second function ƒ2 may be

$$\frac{( V upper - {V}_{b} )}{( V upper - V lower )} .$$

Accordingly, the estimated remaining battery capacity 940 in Phase 2 is equal to ƒ2*BC_{BV}+(1−ƒ2)*BC_{DR}.

In the tertiary phase (see PH3 in FIG. 8), the estimated remaining battery capacity 940 may be based on the voltage 930. In some embodiments, the tertiary phase occurs when the voltage 930 is below the lower battery voltage V_{lower} threshold. As mentioned earlier, the V_{lower} threshold may be between 85-95% of a battery voltage at full battery capacity. For example, the V_{lower} threshold may be between 2.8-2.9V, for a battery with a nominal voltage of about 3V.

In some embodiments, the estimated remaining battery capacity 940 may be computed using a third function ƒ3 of the voltage V_{b}. For example, the third function ƒ3 may be a polynomial function represented by c1*V_{b}⁴+c2*V_{b}³+c3*V_{b}²+c4*V_{b} c5, where c1-c5 may be fitting coefficients determined based on measured and/or simulated data, as explained earlier. It may be understood that the present disclosure is not limited to a particular order of the polynomial and any other polynomial function of the voltage may be used. As an example, the estimated remaining battery capacity 940 in phase PH3 may be calculated as: (6.3800233)V_{b}⁴. V_{b}³+(227.0619483). V_{b}²V_{b}+224.6795938.

In some embodiments, the values of different parameters used for determining the remaining battery capacity may be provided by a manufacturer, measured, designed, assumed, and/or calculated. For example, manufacturer values may provide a self-discharge rate, and a de-rated battery capacity. Measured values may include measurements or may be obtained experimentally. Such experiment-based values may be used when an accurate analytical model is not feasible. For using a measured value in a model, a margin may be added, to allow for measurement errors and for future hardware and/or software changes that could possibly increase power use. Design values may include a value that is part of hardware or software design. Assumed values may include a value assumed (or sometimes estimated) based upon experience or observations, such as how frequently a patient uses their patient remote to connect to the IPG.

It is understood that the above described embodiments in the specification and drawings are, accordingly, to be regarded in an illustrative rather than a restrictive sense. It will, however, be evident that various modifications and changes may be made thereunto without departing from the broader spirit and scope of the disclosure as set forth in the claims.

The use of the terms “a” and “an” and “the” and similar referents in the context of describing the disclosed embodiments (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein and each separate value is incorporated into the specification as if it were individually recited herein. All methods described herein may be performed in any suitable order unless otherwise indicated herein or otherwise clearly contradicted by context. It is to be understood that terms such as “first,” “second,” “third,” etc., do not necessarily limit embodiments of the present disclosure to any particular configuration or orientation. As used herein, the term “about” means+/−10% of the specified value. As used herein, the term “data” is understood to mean one or more values, a set of values, historical values, or a cumulative value of past values. Further, disjunctive language such as the phrase “at least one of x, y, or z,” unless specifically stated otherwise, is intended to be understood within the context as used in general to present that an item may be either x, y or z, or any combination thereof (e.g., x, y and/or Z). Thus, such disjunctive language is not generally intended to, and should not, imply that certain embodiments require each of x, y, and z to be present.

Variations of the embodiments herein may become apparent to those of ordinary skill in the art upon reading the present description. The inventors expect skilled artisans to employ such variations as appropriate and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

What is claimed is:

1. A neurostimulation system comprising: an implantable pulse generator (IPG) that includes a battery, and an external device configured to communicate with the IPG;
wherein the IPG is used over a time period that includes an initial phase and a latter phase;
wherein the external device is configured to receive information from the implantable device including battery energy consumption data and a voltage of the battery;
wherein the external device includes a processor configured to determine an estimate of the capacity of the battery;
wherein, during the initial phase, the processor is configured to determine the estimate of the battery capacity using battery energy consumption data and a battery capacity prior to use of the IPG; and
wherein, during the latter phase, the processor is configured to determine the estimate of the battery capacity based on the battery voltage.

2. The system of claim 1, wherein the time period of use of the IPG includes an intermediate stage between the initial stage and the latter stage, and wherein, during the intermediate phase, the processor is configured to determine the estimate of the battery capacity based on the battery energy consumption data and the battery voltage.

3. The system of claim 2, wherein the battery energy consumption data comprises one or more values associated with battery energy consumed by specific components of the IPG or a cumulative value of the battery energy consumed by the IPG.

4. The system of claim 2, wherein during the intermediate phase, the estimate of battery capacity is determined from a combination of a first battery capacity estimate result based on the battery energy consumption data and a second battery estimate result based on the battery voltage.

5. The system of claim 4, wherein the first and second battery capacity results are combined linearly.

6. The system of claim 4, wherein during the intermediate phase, the first and second battery capacity estimate results are combined linearly such that first battery capacity result based on energy consumption data is fully weighted upon commencement of the intermediate phase and the second battery capacity result based on the voltage is fully weighted at completion of the intermediate phase.

7. The system of claim 4, wherein the intermediate phase occurs between an upper battery voltage threshold and a lower battery voltage threshold.

8. The system of claim 7, wherein the upper battery voltage threshold is between 95%-99% of a nominal voltage of the battery at full charge and the lower battery voltage threshold is between 85%-95% of the nominal voltage of the battery.

9. The system of claim 8, wherein the upper battery voltage threshold is about 2.974 V and the lower battery voltage threshold is 2.870 V when the battery has a nominal voltage of 3.1 V.

10. The system of claim 1, wherein during the latter phase, which is when the voltage is below a lower battery voltage threshold, the estimate is based on the voltage.

11. The system of claim 10, wherein, during the latter phase, the estimate is based on a polynomial function of the voltage that is derived from battery characterization.

12. The system of claim 10, wherein the lower battery voltage threshold is between 85%-95% of a nominal battery voltage of the battery.

13. The system of claim 12, wherein the lower battery voltage threshold is between 2.8-2.9 V, where the nominal voltage is about 3.1 V.

14. The system of claim 1, wherein during the initial phase, the battery capacity estimate comprises subtracting a cumulative amount of energy discharged from the battery from the battery capacity at the beginning of use.

15. The system of claim 14, wherein the battery energy consumption data comprises the cumulative amount of energy discharged from the battery.

16. The system of claim 14, wherein the cumulative amount of energy discharged from the battery is determined by the processor and stored on the IPG.

17. The system of claim 14, wherein the cumulative amount of energy discharged from the battery is determined by the external device and based on the battery energy consumption data received from the IPG.

18. The system of claim 1, wherein the initial phase comprises a first subphase when the battery capacity estimate is above an upper battery capacity threshold and a second subphase below the upper battery capacity threshold, and
wherein in the first subphase, the battery capacity estimate is determined from the battery energy consumption data; and
wherein in the second subphase, the estimate is determined from battery energy consumption data and has a minimum value set at a lower battery capacity threshold.

19. The system of claim 18, wherein the upper battery capacity threshold is about 75% of battery capacity at the beginning of use.

20. The system of claim 18, wherein during the second subphase, the voltage is greater than an upper battery voltage threshold.

21. The system of claim 20, wherein the upper battery voltage threshold is between 95%-99% of a nominal battery voltage of the battery.

22. The system of claim 21, wherein the upper battery voltage threshold is about 2.974 V, wherein the battery has a nominal voltage of about 3.1 V.

23. The system of claim 18, wherein during the second subphase, the estimated battery capacity is less than the upper battery capacity threshold and greater than the lower battery capacity threshold.

24. The system of claim 23, wherein the upper battery capacity threshold is between 70%-75% of a battery capacity at the beginning of use, and the second battery capacity threshold is between 40%-50% of the battery capacity at the beginning of use.

25. The system of claim 1, wherein the battery is a non-rechargeable primary cell battery.

26. The system of claim 25, wherein the battery is a lithium manganese dioxide (Li—MnO2) battery.

27. The system of claim 1, wherein the battery energy consumption data comprises active battery energy consumption data related to stimulation pulse delivery; and fixed battery energy usage data related to fixed energy consumption.

28. The system of claim 27, wherein the fixed battery energy usage data comprises battery energy usage related to at least one or any combination of:
a fixed battery discharge;
a periodic communication with the external device;
one or more housekeeping tasks related to functioning of software within the IPG; and
a quiescent current consumed by the IPG.

29. The system of claim 28, wherein the fixed battery energy usage data set comprises at least one or any combination of:
a first usage data set associated with energy used by a battery self-discharge;
a second usage data set associated with wth energy used by a communication polling at a first scan rate to determine whether the external device is requesting to communicate;
a third usage data set associated with energy used by a radio frequency communication involving a communication with the external device;
a fourth usage data set associated with energy used by a communication at a second scan rate to determine whether the external device is requesting to communicate;
a fifth usage data set associated with energy used by one or more housekeeping tasks;
a sixth usage data set associated with energy used by quiescent current consumed by the IPG;
a seventh usage data set associated with energy used by on-the-shelf-discharge experienced by the battery before connecting to the IPG; and
an eight usage data set associated with energy used by communications with the external device during implanting of the IPG.

30. The system of claim 27, wherein the active usage comprises:
a ninth usage data set associated with an energy usage by the processor for stimulation pulse delivery; and
a tenth usage data set associated with energy used by a stimulation generating circuitry of the IPG for stimulation pulse delivery.

31. An external device communicably coupleable with an implantable device to be positioned in a patient, wherein the implantable device includes a battery, the external device comprising:
a graphical user interface configured to facilitate programming and monitoring of the implantable device; and
a processor coupled with a memory device, wherein the processor is configured to:
establish communication with the implantable device;
receive information from the implantable device including battery energy consumption data and a voltage of the battery; and
determine, based on the received information, an estimate of a capacity of the battery;
wherein the processor is configured to make the determination of the estimate of the capacity of the battery during an initial phase of use of the battery using cumulative battery energy consumption data relative to a total capacity of the battery at the beginning of use of the battery;
wherein the processor is configured to make the determination of the estimate of battery capacity during an intermediate phase of use based on a combination of the cumulative battery energy consumption data set and a voltage of the battery; and
wherein the processor is configured to make the determination of the estimate of the capacity of the battery during a tertiary phase of use based on the voltage of the battery.

32. The external device of claim 31, wherein the cumulative battery energy consumption is the battery energy consumption data received from the implantable device.

33. The external device of claim 31, wherein the cumulative battery energy consumption is determined by the external device from the cumulative battery energy data received from the implantable device, the cumulative battery energy data comprising a data set of one or more values.

34. The external device of claim 31, wherein during the intermediate phase, the battery capacity estimate is determined from a combination of a first battery capacity estimate result based on the battery energy consumption data and a second battery estimate result based on the voltage.

35. The external device of claim 34, wherein the first and second battery capacity results are combined linearly.

36. The external device of claim 34, wherein during the intermediate phase, the first and second battery capacity estimates results are combined linearly such that first battery capacity result based on energy consumption data is fully weighted upon commencement of the intermediate phase and the second battery capacity result based on the voltage is fully weighted at completion of the intermediate phase.

37. The external device of claim 34, wherein the intermediate phase occurs between an upper battery voltage threshold and a lower battery voltage threshold.

38. The external device of claim 37, wherein the upper battery voltage threshold is about 2.974 V and the lower battery voltage threshold is 2.870 V where the battery has a nominal voltage of 3.1 V.

39. The external device of claim 31, wherein during the tertiary phase, when the voltage is below a lower battery voltage threshold, the battery capacity estimate is based on the battery voltage.

40. The external device of claim 39, wherein, during the tertiary phase, the battery capacity estimate is based on a polynomial function of the battery voltage that is derived from battery characterization.

41. The external device of claim 39, wherein the lower battery voltage threshold is between 85-95% of a nominal battery voltage of the battery, wherein when a nominal voltage of the battery is about 3.1 V, the lower battery voltage threshold is between 2.8-2.9 V.

42. The external device of claim 31, wherein during the initial phase, the battery capacity estimate comprises subtracting a cumulative amount of energy discharged from the battery from the battery capacity at the beginning of use of the battery.

43. The external device of claim 31, wherein the initial phase comprises a first subphase when the estimate is above an upper battery capacity threshold and a second subphase below the upper battery capacity threshold, and
wherein in the first subphase, the battery capacity estimate is determined from the battery energy consumption data relative a total capacity;
wherein in the second subphase, the battery capacity estimate is determined from battery energy consumption data relative the total capacity and has a minimum value set at a lower battery capacity threshold.

44. The external device of claim 43, wherein the upper battery capacity threshold is about 75% of battery capacity at the beginning of use and the lower battery capacity is about 46% of the battery capacity at the beginning of use.

45. The external device of claim 43, wherein during the second subphase, the voltage is greater than an upper battery voltage threshold.

46. The external device of claim 45, wherein the upper battery voltage threshold is between 95-99% of a nominal battery voltage of the battery, wherein when a nominal voltage of the battery is about 3.1 V, the upper battery voltage is about 2.974 V.

47. The external device of claim 31, wherein the battery energy consumption data comprises:
an active battery energy consumption data set related to a stimulation pulse delivery; and
a fixed battery energy usage data set related to a fixed energy consumption.

48. The external device of claim 47, wherein the fixed battery energy usage data set comprises battery energy usage related to at least one or any combination of:
a fixed battery discharge;
a periodic communication with the external device;
housekeeping tasks related to functioning of software within the implantable device; and
a quiescent current consumed by the implantable device.

49. A method of determining a battery capacity estimate of a battery of an implantable device over a period of use, the method comprising:
establishing, with an external device, communication with the implantable device;
receiving information from the implantable device including battery energy consumption data and a voltage of the battery; and
determining, based on the received information, the battery capacity estimate, wherein:
during an initial phase of the period of use, the battery capacity estimate is determined from the battery energy consumption data relative to a battery capacity at the beginning of the period of use; and
during a latter phase of the period of use, the battery capacity estimate is based on the battery voltage.

50. The method of claim 49, wherein the latter phase is a final phase of the period of use.

51. The method of claim 49, wherein the period of use includes an intermediate stage between the initial stage and the latter stage, and wherein, during the intermediate phase, the estimate of the battery capacity is determined based on the battery energy consumption data and the battery voltage.
